(19) United States
(12) Patent Application Publication
(10) Pub. No.: US 2020/0000478 A1
(43) Pub. Date: Jan. 2, 2020

(54) ANTIMICROBIAL TOURNIQUET

(71) Applicant: Kaitlin Walden, Indianapolis, IN (US)

(72) Inventor: Kaitlin Walden, Indianapolis, IN (US)

(21) Appl. No.: 16/453,090

(22) Filed: Jun. 26, 2019

Related U.S. Application Data
(60) Provisional application No. 62/691,252, filed on Jun. 28, 2018.

(51) Int. Cl. A61B 17/132; A61B 17/135

(52) U.S. Cl. CPC A61B 17/1322; A61B 2017/12004; A61B 17/135

(57) ABSTRACT

A tourniquet includes a constricting member with a body-contacting portion, a constriction activator to cause the constricting member to constrict around a body part, and an antimicrobial composition on the body-contacting portion of the constricting member. The constricting member may be a strap or a sleeve, and is positioned around a body part to restrict the flow of blood through the body part. The constriction activator causes the constricting member to constrict around a body part. When the constricting member is placed around a body part and the constriction activator is activated, the constricting member constricts around the body part and the antimicrobial composition contacts the body part.

### REFERENCE TO RELATED APPLICATIONS

The present application claims the benefit of U.S. Provisional Patent Application No. 62/691,252, filed Jun. 28, 2018, which is incorporated by reference herein.

### FIELD OF THE INVENTION

The present invention relates generally to first aid devices, and more particularly to tourniquets used to stop arterial bleeding.

### BACKGROUND TO THE INVENTION

Tourniquets have long been used in emergency situations to stop arterial blood flow, particularly on the limbs. Conventional tourniquets include a strap and a rod that may be used to twist the strap to constrict it around a person's arm or leg. A blocking structure may be included to hold the twisted rod in its constricted position to prevent unwanted release of the pressure applied by the strap. More modern tourniquets include an inflatable cuff that is inflated by a pneumatic pump to expand the cuff and stop blood flow.

Infections are known to arise from the wounds that necessitated the tourniquet's use. A need therefore exists for a tourniquet that stops arterial bleeding whilst also minimizing would infections. The present invention addresses that need.

### SUMMARY OF THE INVENTION

A tourniquet for restricting a flow of blood in a body part includes:

(a) a constricting member effective for being positioned around a body part and for being constricted to restrict the flow of blood through the body part, said constricting member having a body-contacting portion;

(b) a constriction activator adapted to cause the constricting member to constrict around a body part; and

(c) an antimicrobial composition on the body-contacting portion of the constricting member;

wherein the tourniquet is adapted such that when the constricting member is placed around a body part and the constriction activator is activated, the constricting member constricts around the body part and the antimicrobial composition contacts the body part.

The constricting member may be a strap, and the constriction activator may be a rod. When the rod is rotated at least a portion of the strap is twisted, shortening its effective length and constricting the strap around a body part.

The constricting member may be a sleeve, such as an inflatable cuff, and the constriction activator may comprise a pneumatic device. The pneumatic device may pump air into the inflatable cuff, thereby causing the inside opening in the cuff to constrict around the body part, and further causing the antimicrobial composition to contact the body part.

The antimicrobial composition may comprise at least one antibacterial agent.

### DESCRIPTION OF THE DRAWINGS

FIGS. 1A and 1B show one embodiment of the present invention, wherein the tourniquet comprises a strap that is tightened by a rod.

FIG. 2 shows another embodiment of the present invention, wherein the tourniquet comprises an inflatable cuff that is constricted by a pneumatic pump.

FIG. 3 shows antimicrobial wipes that may be provided for use with certain embodiments of the present invention.

FIG. 4 shows various forms of tourniquets that may be used to make the antimicrobial tourniquets of the present invention.

FIG. 5A shows one embodiment of the present invention, wherein the tourniquet comprises a strap that is tightened by a rod.

FIG. 5B shows one embodiment of a pouch that may be used to contain antimicrobial wipes in certain embodiments of the present invention.

FIG. 5C shows one embodiment of an antimicrobial sheet that may be used in certain embodiments of the present invention.

FIG. 5D shows one embodiment of the inventive tourniquet being used on a patient.

### DESCRIPTION OF THE PREFERRED EMBODIMENTS

For the purposes of promoting an understanding of the principles of the invention, reference will now be made to certain embodiments and specific language will be used to describe the same. It will nevertheless be understood that no limitation of the scope of the invention is thereby intended, with such alterations and modifications to the illustrated device being contemplated as would normally occur to one skilled in the art to which the invention relates.

As indicated above, one aspect of the present invention provides a tourniquet for restricting a flow of blood in a body part. The tourniquet preferably includes:

(a) a constricting member effective for being positioned around a body part and for being constricted to restrict the flow of blood through the body part, said constricting member having a body-contacting portion;

(b) a constriction activator adapted to cause the constricting member to constrict around a body part; and

(c) an antimicrobial composition on the body-contacting portion of the constricting member.

The tourniquet is adapted such that when the constricting member is placed around a body part and the constriction activator is activated, the constricting member constricts around the body part and the antimicrobial composition contacts the body part.

The constricting member may be a strap, and the constriction activator may be a rod. When the rod is rotated at least a portion of the strap is twisted, shortening its effective length and constricting the strap around a body part.

The constricting member may be a sleeve, such as an inflatable cuff, and the constriction activator may comprise a pneumatic device. The pneumatic device may pump air into the inflatable cuff, thereby causing the inside opening in the cuff to constrict around the body part, and further causing the antimicrobial composition to contact the body part.

The antimicrobial composition is effective to reduce and/or eliminate deadly infection causing microbes. The antimicrobial composition may comprise at least one antibacterial agent.

In one embodiment the inventive tourniquet has a constricting member that is impregnated with an antimicrobial composition. When the tourniquet is tightened on a patient's arm or leg, the antimicrobial composition contacts the wound and releases antimicrobial medicament to the wound area.

In another embodiment the tourniquet has a constricting member made of conventional material, and the antimicrobial composition is provided separately. For example, the antimicrobial composition may be provided on sheets that are placed over the wound before the tourniquet is tightened.

When antimicrobial sheets are used, such sheets may be stored in a pocket that is fixed to the tourniquet. The sheets may be single-use sheets, or they may be reusable.

The antimicrobial sheets may be adapted to be fixed to a patient's wounds in difficult environments. For example, the sheets may include water resistance and may be adapted for easy wash and go.

It is to be appreciated that one goal of the invention is to reduce loss of life and increase positive outcome from mass causality, trauma, and war when people are not able to be attended to quickly. The inventive antimicrobial tourniquets may also be used on regular basis as infection control protocol in every day EMS.

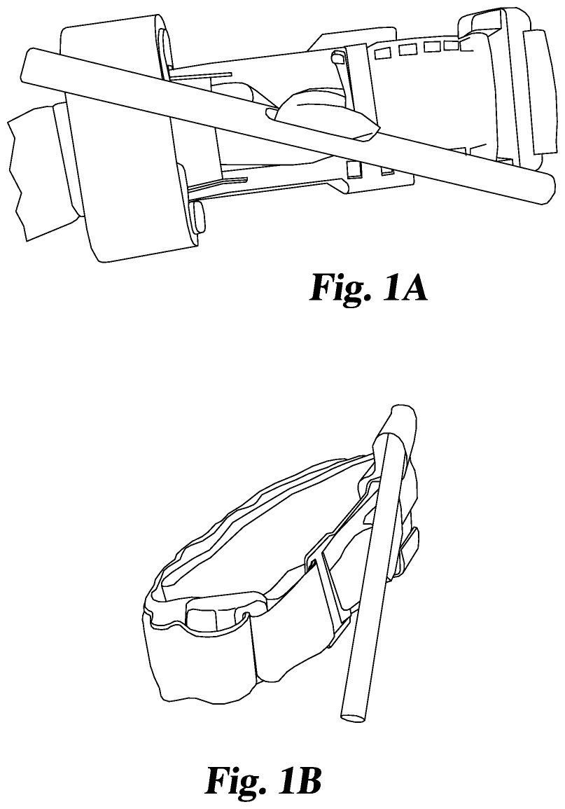

Referring now to the drawings, FIG. 1 shows one embodiment of the inventive antimicrobial tourniquet. Tourniquet 10 includes a constricting member 11 effective for being positioned around a body part and for being constricted to restrict the flow of blood through the body part. Constricting member 11 has a body-contacting portion 12. Tourniquet 10 also includes a constriction activator 13 adapted to cause the constricting member to constrict around a body part. An antimicrobial composition 14 is provided on the body-contacting portion 12 of constricting member 11. In the illustrated embodiment constricting member 11 comprises a strap, and constriction activator 13 comprises a rod.

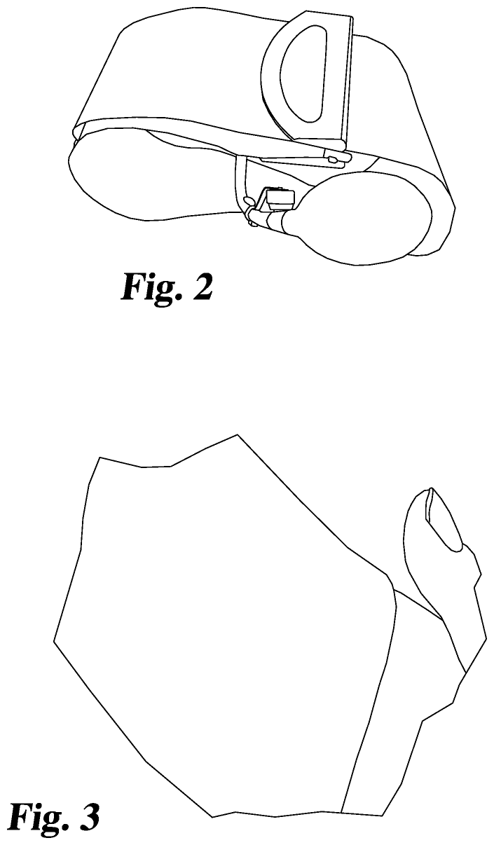

FIG. 2 shows one embodiment of the inventive antimicrobial tourniquet. Tourniquet 20 includes a constricting member 21 effective for being positioned around a body part and for being constricted to restrict the flow of blood through the body part. In the illustrated embodiment constricting member comprises an inflatable cuff 21 that includes a body-contacting portion 22. Tourniquet 20 also includes a constriction activator 23 adapted to cause the constricting member to constrict around a body part. In the illustrated embodiment constriction activator 23 comprises a pneumatic device effective for inflating said inflatable cuff. An antimicrobial composition 24 is provided on the body-contacting portion 22 of constricting member 21.

In some embodiments the antimicrobial composition is embedded in the constricting member as described and shown herein. In other embodiments the antimicrobial composition is provided on a separate material, such as a sheet material, that may be placed between the constricting member and the body part so that when the constricting member is constricted around the body part the antimicrobial composition contacts the body part.

In some embodiments the antimicrobial composition may comprise sulfacetamide sodium, and/or silver sulfadiazine, and/or bacitracin, and/or neomycin, and/or polymyxin B, and/or erythromycin, and/or zinc sulfate, and/or copper sulfate.

FIG. 3 shows one embodiment of the antimicrobial wipes that may be provided in a pocket of the antimicrobial tourniquet. The antimicrobial wipes may include one or more of the antimicrobial compositions identified above,

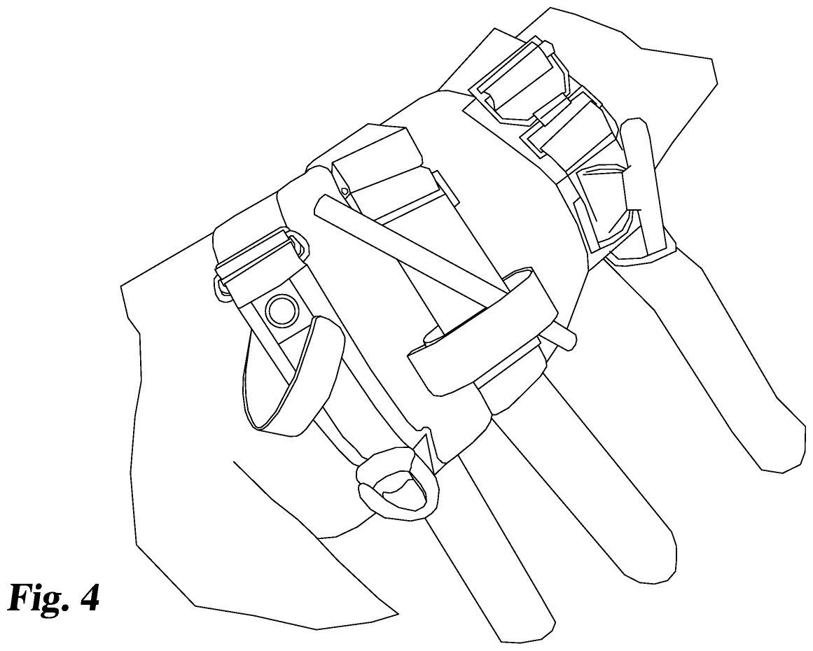

FIG. 4 shows various forms of tourniquets that may be used to make the antimicrobial tourniquets of the present invention.

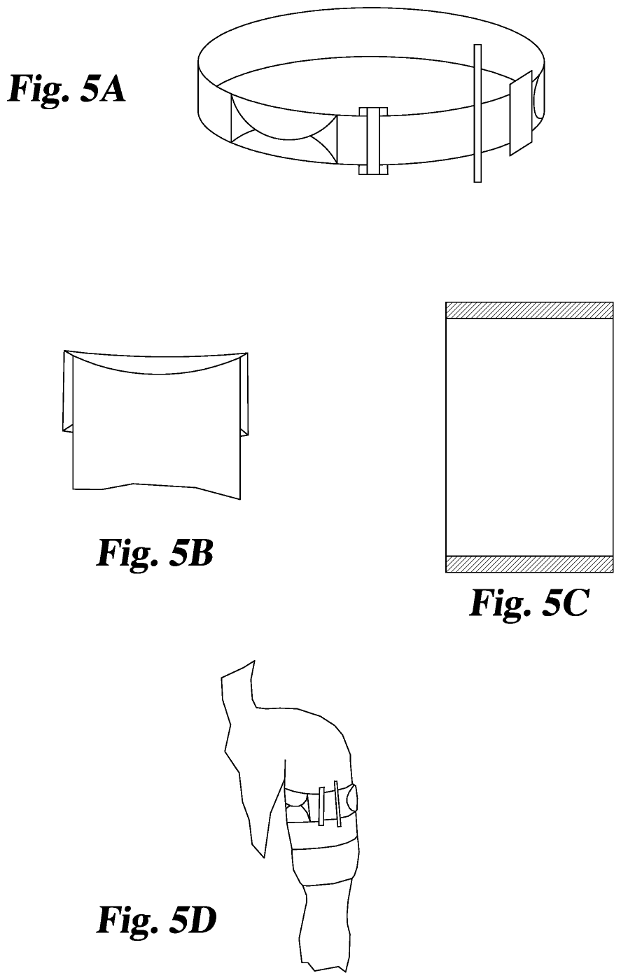

FIG. 5 shows additional inventive elements, such as the antimicrobial sheets that may be provided in slip pockets for use with the antimicrobial tourniquets of the present invention.

While the invention has been illustrated and described in detail in the drawings and foregoing description, the same are to be considered as illustrative and not restrictive, it being understood that only certain preferred embodiments have been shown and described and that all changes and modifications that come within the spirit of the invention are desired to be protected. In addition, it is to be appreciated that the present invention may comprise or consist essentially of any or all of the illustrated or described features. For example, the present invention includes devices comprising each of the embodiments described above and/or illustrated in the drawings, and the present invention includes devices consisting essentially of any of the embodiments described above and/or illustrated in the drawings. Additionally, any or all of the features and/or embodiments disclosed herein may be combined with any or all of the other features and/or embodiments disclosed herein to provide a device that comprises or consists essentially of such features.

Finally, the phrase A “and/or” B is used in this disclosure to mean A alone, or B alone, or both A and B.

1. A tourniquet for restricting a flow of blood in a body part, the tourniquet comprising:
(a) a constricting member effective for being positioned around a body part and for being constricted to restrict the flow of blood through the body part, said constricting member having a body-contacting portion;
(b) a constriction activator adapted to cause the constricting member to constrict around a body part; and
(c) an antimicrobial composition on the body-contacting portion of the constricting member;
wherein the tourniquet is adapted such that when the constricting member is placed around a body part and the constriction activator is activated, the constricting member constricts around the body part and the antimicrobial composition contacts the body part.

2. The tourniquet of claim 1 wherein said constricting member is a strap and said constriction activator is a rod adapted to be rotated, such that when the rod is rotated at least a portion of the strap is twisted, shortening its effective length and constricting the strap around a body part.

3. The tourniquet of claim 1 wherein said constricting member is a sleeve and said constriction activator comprises a pneumatic device.

4. The tourniquet of claim 3 wherein said constricting member is an inflatable cuff and said constriction activator comprises a pneumatic device effective for inflating said inflatable cuff.

5. The tourniquet of claim 1 wherein said antimicrobial composition comprises at least one antibacterial agent.

6. A tourniquet for restricting a flow of blood in a body part, the tourniquet comprising:
(a) a strap effective to constrict around a body part to restrict the flow of blood therethrough, said strap having a body-contacting portion;
(b) a rod adapted to be rotated such that when the rod is rotated at least a portion of the strap is twisted, shortening its effective length and constricting the strap around a body part; and
(c) an antimicrobial composition on the body-contacting portion of the strap;
wherein said tourniquet is adapted such that when the strap is placed around a body part and the rod is rotated, the strap constricts around the body part and the antimicrobial composition contacts the body part.

7. A tourniquet for restricting a flow of blood in a body part, the tourniquet comprising:
(a) a sleeve effective to constrict around a body part to restrict the flow of blood therethrough, said strap having a body-contacting portion;
(b) a sleeve constrictor adapted to constrict the sleeve around a body part; and
(c) an antimicrobial composition on the body-contacting portion of the sleeve;
wherein said tourniquet is adapted such that when the sleeve is placed around a body part and the sleeve constrictor is activated, the sleeve constricts around the body part and the antimicrobial composition contacts the body part.
